(19) United States
(12) Patent Application Publication
(10) Pub. No.: US 2026/0090779 A1
(43) Pub. Date: Apr. 2, 2026

(54) X-RAY CT APPARATUS, MEDICAL IMAGE PROCESSING APPARATUS, METHOD, AND STORAGE MEDIUM

(71) Applicant: CANON MEDICAL SYSTEMS CORPORATION, Tochigi (JP)

(72) Inventors: Kyotaro HORIO, Nasushiobara (JP); Takahiko NISHIOKA, Otawara (JP); Haruka KUNEZAKI, Utsunomiya (JP); Sumika KAWASAKA, Nasushiobara (JP); Daiki ANDO, Ichikawa (JP)

(73) Assignee: CANON MEDICAL SYSTEMS CORPORATION, Tochigi (JP)

(21) Appl. No.: 19/340,261

(22) Filed: Sep. 25, 2025

(30) Foreign Application Priority Data
Sep. 27, 2024 (JP) 2024-169336

(51) Int. Cl. A61B 6/00; A61B 6/03; G06T 7/00; G06T 7/11

(52) U.S. Cl. CPC A61B 6/5211; A61B 6/032; G06T 7/0012; G06T 7/11; G06T 2207/10081; G06T 2207/20076; G06T 2207/30056

(57) ABSTRACT

An X-ray CT apparatus according to an embodiment includes processing circuitry. The processing circuitry acquires information on a structure serving as a target for segmentation. The processing circuitry acquires information on an existence probability of a structure in a CT image on the basis of the information on the structure. The processing circuitry sets a segmentation region for performing segmentation of the structure on a part of the CT image on the basis of information on the existence probability of the structure. The processing circuitry performs segmentation of the structure on a segmentation region in the CT image.

### CROSS-REFERENCE TO RELATED APPLICATIONS

This application is based upon and claims the benefit of priority from Japanese Patent Application No. 2024-169336, filed on Sep. 27, 2024; the entire contents of which are incorporated herein by reference.

### FIELD

Embodiments disclosed herein and in the drawings relate to an X-ray CT apparatus, a medical image processing apparatus, a method, and a storage medium.

### BACKGROUND

Conventionally, segmentation has been used to detect a structure serving as a target in a medical image for analysis of a disease and other conditions using a medical image. For example, segmentation is performed to detect a structure serving as a target from a medical image using a method based on machine learning techniques, including deep learning, or known image processing techniques such as Otsu's binarization method based on CT values. Such segmentation detecting a structure serving as a target from a medical image is important for volume measurement of the structure and for detailed analysis at a later stage.

### BRIEF DESCRIPTION OF THE DRAWINGS

FIG. 1 is a diagram illustrating a configuration example of a medical image processing apparatus according to a first embodiment;

FIG. 2 is a flowchart illustrating a process procedure performed by the medical image processing apparatus according to the first embodiment;

FIG. 3A is a diagram illustrating an example of processing executed by a control function according to the first embodiment;

FIG. 3B is a diagram illustrating an example of the processing executed by a control function according to the first embodiment;

FIG. 4 is a diagram illustrating an example of the segmentation result according to the first embodiment;

FIG. 5A is a diagram illustrating an example of processing executed by the medical image processing apparatus according to the first embodiment;

FIG. 5B is a diagram illustrating an example of the processing executed by the medical image processing apparatus according to the first embodiment;

FIG. 6A is a diagram illustrating an example of setting a segmentation region using a probability map according to the first embodiment;

FIG. 6B is a diagram illustrating an example of setting a segmentation region using the probability map according to the first embodiment;

FIG. 6C is a diagram illustrating an example of setting a segmentation region using the probability map according to the first embodiment;

FIG. 7 is a diagram illustrating an example of display according to the first embodiment;

FIG. 8 is a diagram illustrating an example of display of the segmentation result according to the first embodiment; and

FIG. 9 is a diagram illustrating a configuration example of an X-ray CT apparatus according to another embodiment.

### DETAILED DESCRIPTION

An X-ray CT apparatus according to an embodiment includes processing circuitry. The processing circuitry is configured to acquire information on a structure serving as a target for segmentation. The processing circuitry is configured to acquire information on an existence probability of a structure in a CT image on the basis of the information on the structure. The processing circuitry is configured to set a segmentation region for performing segmentation of the structure on a part of the CT image on the basis of information on the existence probability of the structure.

The processing circuitry is configured to perform segmentation of the structure on a segmentation region in the CT image.

Hereinafter, with reference to the drawings, embodiments of an X-ray CT apparatus, a medical image processing apparatus, method, and a computer program will be described in detail. The X-ray CT apparatus, the medical image processing apparatus, the method, and the computer program according to the present application are not limited to the embodiments described below.

### First Embodiment

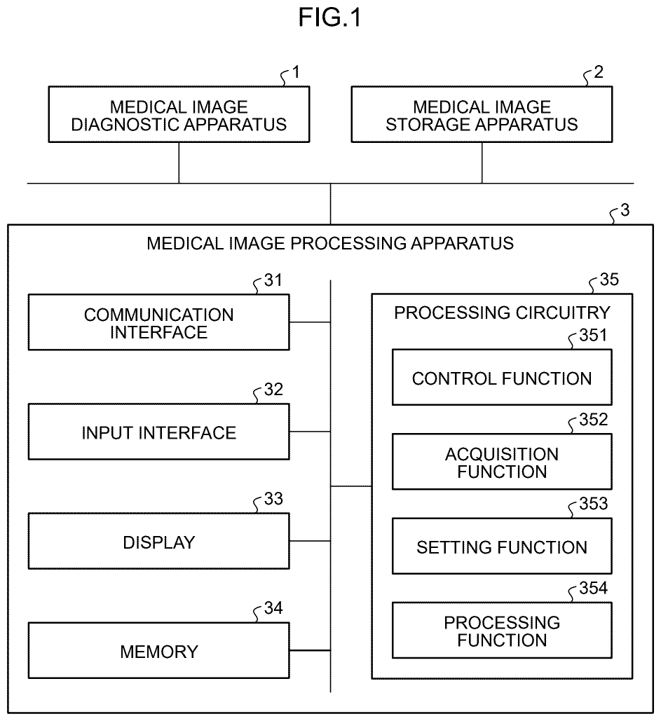

FIG. 1 is a diagram illustrating a configuration example of a medical image processing apparatus according to a first embodiment. For example, as illustrated in FIG. 1, a medical image processing apparatus 3 according to the present embodiment is communicably connected to a medical image diagnostic apparatus 1 and a medical image storage apparatus 2 via a network. Various other apparatuses and systems may be connected to the network illustrated in FIG. 1.

The medical image diagnostic apparatus 1 images an image of a subject to generate a medical image. The medical image diagnostic apparatus 1 then transmits the generated medical image to various apparatuses on the network. Examples of the medical image diagnostic apparatus 1 include an X-ray diagnostic apparatus, an X-ray computed tomography (CT) apparatus, a magnetic resonance imaging (MRI) apparatus, an ultrasound diagnostic apparatus, a single photon emission computed tomography (SPECT) apparatus, and a positron emission computed tomography (PET) apparatus.

The medical image storage apparatus 2 stores various medical images related to the subject. Specifically, the medical image storage apparatus 2 receives a medical image from the medical image diagnostic apparatus 1 via the network, and stores and retains the medical image in its own internal memory. For example, the medical image storage apparatus 2 is implemented by a computer device such as a server or workstation. In addition, for example, the medical image storage apparatus 2 is implemented by a picture archiving and communication system (PACS) or the like, and stores medical images in a format compliant with digital imaging and communications in medicine (DICOM).

The medical image processing apparatus 3 performs various types of information processing on medical images collected from the subject. Specifically, the medical image processing apparatus 3 receives medical images from the medical image diagnostic apparatus 1 or the medical image storage apparatus 2 via a network and performs various types of information processing using the medical images. For example, the medical image processing apparatus 3 is implemented by a computer device such as a server or workstation.

For example, the medical image processing apparatus 3 has a communication interface 31, an input interface 32, a display 33, a memory 34, and processing circuitry 35.

The communication interface 31 controls transmission and communication of various data transmitted and received between the medical image processing apparatus 3 and other apparatuses connected via the network. Specifically, the communication interface 31 is connected to the processing circuitry 35 and transmits data received from the other apparatuses to the processing circuitry 35, or transmits data received from the processing circuitry 35 to the other apparatuses. For example, the communication interface 31 is implemented by a network card, a network adapter, a network interface controller (NIC), or the like.

The input interface 32 receives various instructions and input operations of various pieces of information from a user. Specifically, the input interface 32 is connected to the processing circuitry 35, converts the input operations received from the user into electrical signals, and outputs the electrical signals to the processing circuitry 35. For example, the input interface 32 is implemented by a trackball, a switch button, a mouse, a keyboard, a joystick, a touch pad for input operations in response to a touch on an operation surface, a touch screen in which a display screen and a touch pad are integrated, a non-contact input interface using an optical sensor, a voice input interface, or the like. In the present specification, the input interface 32 is not limited only to those with physical operating components such as a mouse and a keyboard. For example, an electrical signal processing circuit that receives electrical signals corresponding to input operations input from an external input device provided separately from the apparatus and that transmits these electrical signals to a control circuit is also an example of the input interface 32.

The display 33 displays various pieces of information and data. Specifically, the display 33 is connected to the processing circuitry 35 and displays various pieces of information and data received from the processing circuitry 35. For example, the display 33 is implemented by a liquid crystal display, a cathode ray tube (CRT) display, a touch panel, a light emitting diode (LED) display, or the like.

The memory 34 stores various data and various computer programs. Specifically, the memory 34 is connected to the processing circuitry 35 and stores data received from the processing circuitry 35, or reads stored data and transmits the data to the processing circuitry 35. For example, the memory 34 is implemented by a semiconductor memory device such as random access memory (RAM) or flash memory, or a hard disk, an optical disk, or the like.

The processing circuitry 35 controls the entire medical image processing apparatus 3. For example, the processing circuitry 35 performs various processes in response to input operations received from the user via the input interface 32. For example, the processing circuitry 35 receives data transmitted by the other apparatuses via the communication interface 31 and stores the received data in the memory 34. For example, the processing circuitry 35 transmits data received from the memory 34 to the communication interface 31, thereby transmitting the data to the other apparatuses. For example, the processing circuitry 35 displays the data received from the memory 34 on the display 33.

As described above, the configuration example of the medical image processing apparatus 3 according to the present embodiment has been described. For example, the medical image processing apparatus 3 according to the present embodiment is installed in medical facilities such as hospitals and clinics to support various diagnoses and treatment plans made by users such as physicians. Specifically, in the segmentation processing for medical images, the medical image processing apparatus 3 performs local segmentation on the basis of information on a structure desired by a user.

As described above, methods based on machine learning technologies and image processing technologies have been applied to segmentation of medical images, but these methods may not be able to efficiently perform segmentation with high accuracy. For example, in a case where deep learning is used to perform segmentation for each of a plurality of different structures, a large amount of computational resources is required during training, and inference during segmentation is a separate process for each structure, resulting in an increase in processing time. In addition, in a case where segmentation of the structures is performed using multi-class inference based on deep learning, it may not be possible to extract an independent region within the same class (for example, a fractured region). Furthermore, in a case where segmentation is performed on the basis of pixel values in a medical image, it may not be possible to classify structures in a series of structures with similar pixel values.

Therefore, in the present embodiment, high-accuracy segmentation can be efficiently performed by executing local segmentation on the basis of information regarding a structure desired by the user. Hereinafter, a detailed description of the medical image processing apparatus 3 with this configuration will be described.

For example, as illustrated in FIG. 1, in the present embodiment, the processing circuitry 35 of the medical image processing apparatus 3 executes a control function 351, an acquisition function 352, a setting function 353, and a processing function 354. Here, the processing circuitry 35 is an example of processing circuitry.

The control function 351 generates and controls various graphical user interfaces (GUIs) and various pieces of display information to be displayed on the display 33 in response to operations via the input interface 32. For example, the control function 351 causes the display 33 to display the results of processing executed by each function. The control function 351 also acquires medical images of the subject from the medical image diagnostic apparatus 1 or medical image storage apparatus 2 via the communication interface 31. Specifically, the control function 351 acquires medical images that contain three-dimensional or two-dimensional morphological information. The control function 351 acquires CT images, ultrasound images, MRI images, X-ray images, and the like as medical images described above.

The control function 351 also acquires information on a structure serving as a target for segmentation, and this processing will be described in detail later.

The acquisition function 352 acquires information on the existence probability of a structure in a medical image on the basis of information on the structure. The processing executed by the acquisition function 352 will be described in detail later.

The setting function 353 sets a segmentation region for performing segmentation of the structure on a part of the medical image on the basis of the information on the existence probability of the structure. The processing executed by the setting function 353 will be described in detail later.

The processing function 354 performs segmentation of the structure with respect to the segmentation region of the medical image. The processing executed by the processing function 354 will be described in detail later.

The processing circuitry 35 described above is implemented by, for example, a processor. In this case, each of the above-described processing functions is stored in the memory 34 in the form of a computer program executable by a computer. The processing circuitry 35 reads each computer program from the memory 34 and executes the computer program to implement a function corresponding to each computer program. In other words, the processing circuitry 35, in a state where each of the computer programs has been read out, has each of the processing functions illustrated in FIG. 1.

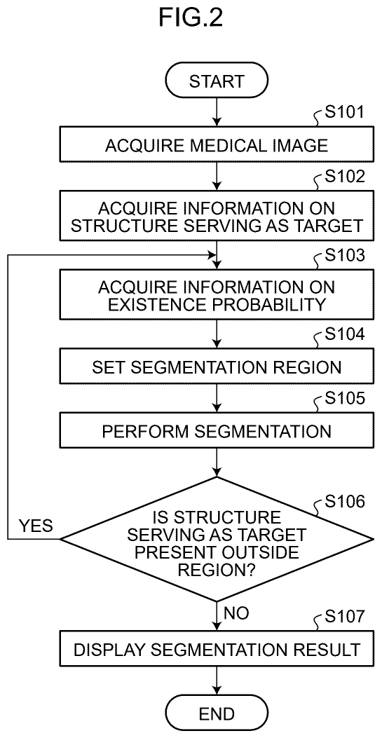

Next, the process procedure performed by the medical image processing apparatus 3 are described using FIG. 2, followed by a detailed description of each process. FIG. 2 is a flowchart illustrating a processing procedure performed by the medical image processing apparatus 3 according to the first embodiment.

For example, as illustrated in FIG. 2, in the present embodiment, the control function 351 acquires a medical image of a subject from the medical image diagnostic apparatus 1 or medical image storage apparatus 2 (step S101) and acquires information on a structure serving as a target for segmentation (step S102). For example, these processes are implemented by the processing circuitry 35 calling, from the memory 34, and executing the computer program corresponding to the control function 351.

Subsequently, the acquisition function 352 acquires information on an existence probability of the structure serving as a target for segmentation in the medical image (step S103). For example, this process is implemented by the processing circuitry 35 calling, from the memory 34, and executing the computer program corresponding to the acquisition function 352.

Subsequently, the setting function 353 sets a segmentation region with respect to a part of the medical image (step S104). For example, this process is implemented by the processing circuitry 35 calling, from the memory 34, and executing the computer program corresponding to the setting function 353.

Subsequently, the processing function 354 performs segmentation (step S105). For example, this process is implemented by the processing circuitry 35 calling, from the memory 34, and executing the computer program corresponding to the processing function 354.

Subsequently, the acquisition function 352 determines whether or not the structure serving as a target is present outside the segmentation region (step S106). For example, this process is implemented by the processing circuitry 35 calling, from the memory 34, and executing the computer program corresponding to the acquisition function 352.

Here, in a case where the structure serving as a target is present outside the region (Yes at step S106), the acquisition function 352 returns to step S103 to execute the process.

On the other hand, in a case where the structure serving as a target is not present outside the region (No at step S106), the control function 351 allows displaying of a segmentation result (step S107). For example, these processes are implemented by the processing circuitry 35 calling, from the memory 34, and executing the computer program corresponding to the control function 351.

Hereinafter, each process executed by the medical image processing apparatus 3 will be described in detail.

### Process for Acquiring Medical Image

As described with step S101 in FIG. 2, the control function 351 acquires the medical image containing three-dimensional or two-dimensional morphological information in response to an operation to acquire the medical image via the input interface 32. For example, the control function 351 acquires a CT image (volume data) imaged in three dimensions. The medical image acquired herein is not limited to a 3D CT image, and can be any medical image as long as it is a medical image serving as a target for segmentation. In addition, medical images in the present embodiment include raw data collected by the medical image diagnostic apparatus 1, image data after reconstruction of the raw data, and images for which various types of image processing are performed on the image data.

### Process for Acquiring Information on Structure Serving as Target

As described with step S102 in FIG. 2, the control function 351 acquires information on the acquired medical image regarding the structure serving as a target for segmentation. For example, the control function 351 acquires the structure serving as a target for segmentation on the basis of a designation operation by an operator on the medical image. In such cases, the control function 351 generates a display image from the acquired medical image and causes the display 33 to display the generated display image.

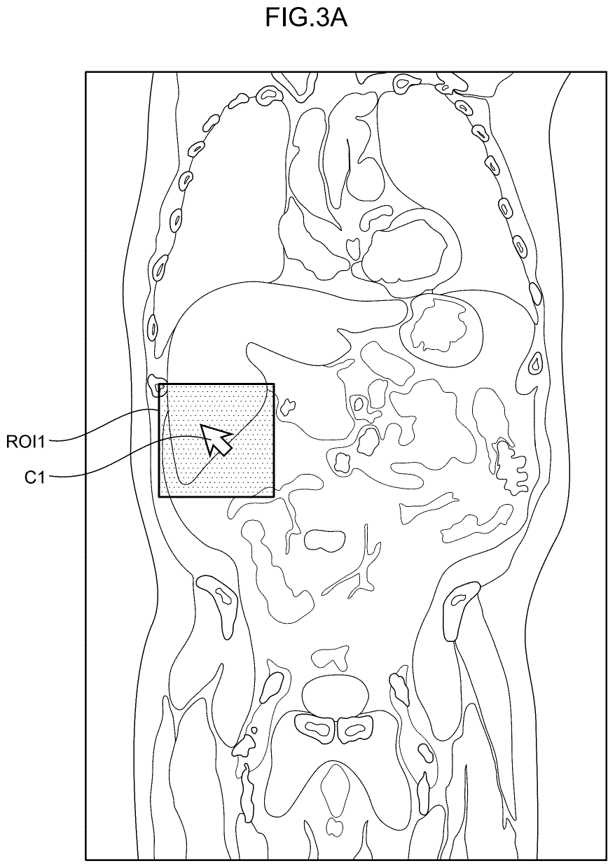

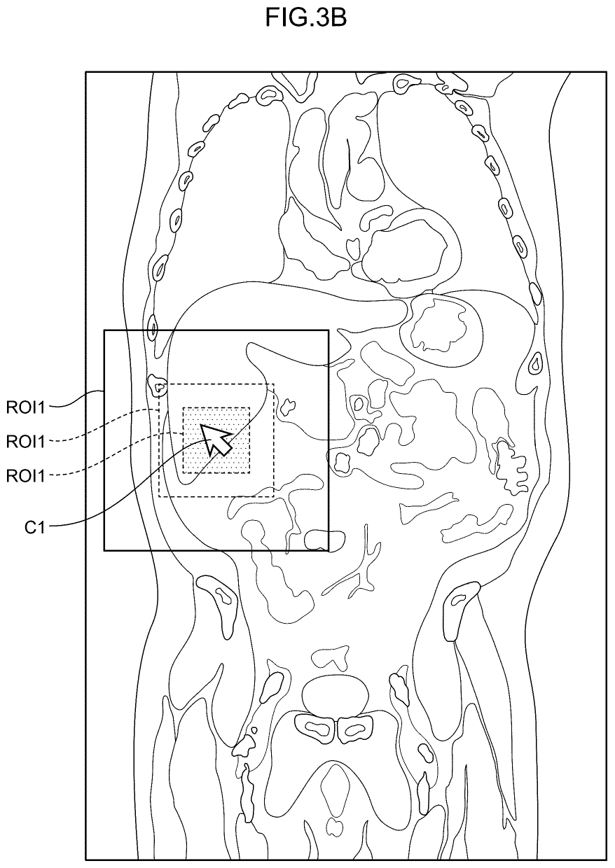

The control function 351 acquires the structure corresponding to the specified position on the display image displayed on the display 33 as the structure serving as a target for segmentation. Hereinafter, an example of processing executed by the control function 351 will be described with reference to FIGS. 3A and 3B. FIGS. 3A and 3B are diagrams each illustrating an example of the processing executed by the control function 351 according to the first embodiment.

For example, as illustrated in FIG. 3A, the control function 351 generates a display image from the acquired three-dimensional CT image (volume data) and causes the display 33 to display the image, as well as a cursor C1 to specify the structure serving as a target for segmentation. Here, as illustrated in FIG. 3A, a segmentation region ROI1 that follows the movement of the cursor C1 is associated with the cursor C1. In other words, in a case where the operator operates the input interface 32 to move the cursor C1, the segmentation region ROI1 also follows and moves.

The operator operates the input interface 32 to move the cursor C1 to the position of the structure serving as a target for segmentation and performs an operation (for example, mouse click operation) to determine the target structure. The control function 351 acquires the structure serving as a target for segmentation by receiving the above-described operation by the operator. Furthermore, the processing function 354 acquires the segmentation region ROI1 disposed on the image by the above-described operations by the operator as a region where segmentation is to be performed. In FIG. 3A, the region where segmentation is performed is illustrated in a two-dimensional display image, but the segmentation is performed in three dimensions.

In other words, in a case where the segmentation region ROI1 is set by the operator, the segmentation region ROI1 is set as the segmentation region at step S104, regardless of the information on the existence probability acquired at step S103.

Here, the size, shape, orientation, and the like of the segmentation region ROI1 associated with the cursor C1 can be changed optionally by the operator. For example, the control function 351 changes the size of the segmentation region ROI1 by receiving the mouse wheel operation by the operator, as illustrated in FIG. 3B. The operator can set a region serving as a target for segmentation within a part of the medical image by changing the size of the segmentation region ROI1 to include the structure serving as a target for segmentation. However, the larger the segmentation region, the greater the processing load involved in the segmentation.

Therefore, in the present embodiment, the segmentation region can be controlled so as not to become excessively large by employing a configuration in which a plurality of sub-regions constitute the segmentation region, and the sub-regions are arranged on the basis of the existence probability of the structure serving as a target for segmentation.

In such cases, for example, as illustrated in FIG. 3A, the segmentation region ROI1 is set at a size that includes part of the structure (liver) serving as a target for segmentation and is used for the structure designation operation. In a case where the segmentation region ROI1 is set at the size illustrated in FIG. 3A, the segmentation region ROI1 is a small region that forms a part of the entire segmentation region.

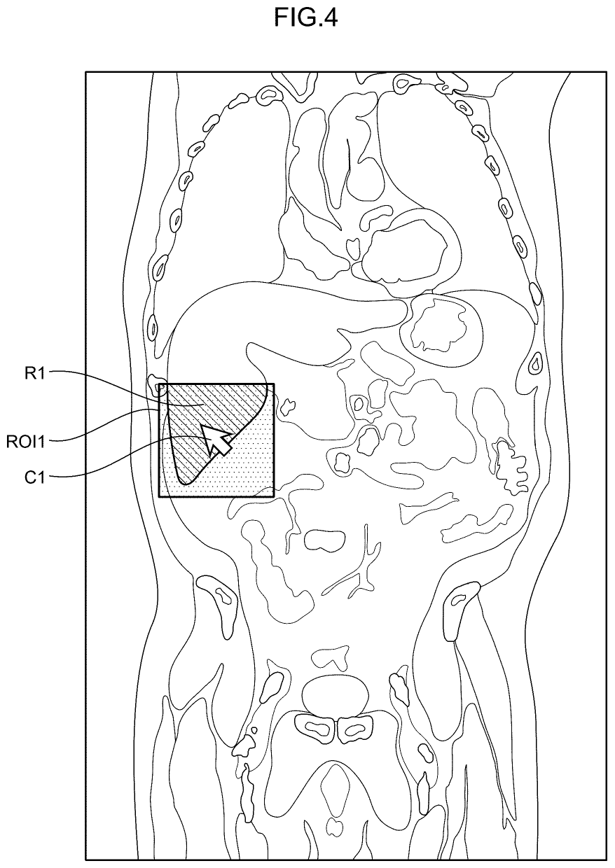

In a case where the segmentation region ROI1 is set by the operator at the position illustrated in FIG. 3A, a region R1 illustrated in FIG. 4 is extracted in the segmentation process at step S105. FIG. 4 is a diagram illustrating an example of the segmentation result according to the first embodiment.

### Process for Acquiring Information on Existence Probability

As described with step S103 in FIG. 2, the acquisition function 352 acquires information on the existence probability of the structure. Specifically, the acquisition function 352 acquires at least one of structure continuity, a probability map, or anatomical landmarks as information on the existence probability of the structure.

For example, in a case of acquiring the structure continuity as information on the existence probability, the acquisition function 352 acquires the structure continuity specified by the operator (indicated by the cursor C1) or the continuity with the region extracted by the segmentation process. In one example, the acquisition function 352 acquires the pixel values of the specified structure (or the region extracted by the segmentation process) and the surrounding pixel values. In other words, the acquisition function 352 acquires pixel values as information for determining whether or not the structure is connected (whether or not the structure is continuous) across the inside and outside of the segmentation region.

For example, in a case of acquiring a probability map as information on the existence probability, the acquisition function 352 acquires a probability map output in segmentation based on deep learning. In one example, the acquisition function 352 acquires the probability map output in the segmentation at step S105. Here, the probability map indicates a per-pixel class probability. In other words, the probability map of the structure serving as a target for segmentation is information indicating whether or not each pixel in the medical image is the target structure.

For example, the acquisition function 352 acquires a probability map represented by continuous values in the range of [0, 1]. The acquisition function 352 is not only capable of acquiring the probability map output in the process at step S105, but also acquiring the probability map, by inputting the medical image acquired by the control function 351, into a trained model formed through deep learning.

Here, the acquisition function 352 can acquire, as the probability map, at least one of a probability map of the structure or a probability map of structures other than the structure. In other words, the acquisition function 352 can acquire the probability of the structure serving as a target for segmentation and the probability of a structure other than the structure serving as a target for segmentation.

For example, in a case of acquiring anatomical landmarks as information on the existence probability, the acquisition function 352 detects anatomical landmarks of the human body by performing image processing, such as pattern recognition, on the medical image acquired by the control function 351. The acquisition function 352 associates identifiers, each uniquely specifying anatomical landmarks, with pixels corresponding to the anatomical landmarks in the medical image, and stores the identifiers. It is possible to estimate the positional relationship among organs in the medical image.

### Process for Setting Segmentation

As described with step S104 in FIG. 2, the setting function 353 sets the segmentation region on the basis of the information on the existence probability. Specifically, the setting function 353 sets the segmentation region for a region where the structure serving as a target for segmentation is likely to be present. For example, as illustrated in FIG. 4, in a case where a sub-region of an initial segmentation region is set by the operator and segmentation is executed to extract the region R1, the setting function 353 sets a segmentation region for a region that is highly likely to be connected to the region R1. Here, the setting function 353 estimates the region that is highly likely to be connected to the region R1 on the basis of the information on the existence probability of the structure acquired by the acquisition function 352.

For example, in a case where the structure continuity is acquired as the information on the existence probability, the setting function 353 sets segmentation regions for the region R1 and the contiguous region. In one example, the setting function 353 compares the pixel values of the region R1 with the surrounding pixel values and sets segmentation regions in a direction of pixels having pixel values similar to the pixel values of the region R1.

For example, in a case where a probability map has been acquired as information on the existence probability, the setting function 353 sets a segmentation region in a direction where the probability of being the region R1 increases.

For example, in a case where the anatomical landmarks are obtained as information on the existence probability, the setting function 353 estimates the arrangement state of the organ corresponding to the region R1 on the basis of the anatomical landmarks. The setting function 353 then sets, on the basis of the estimated arrangement information, a segmentation region for a region in the medical image where the organ corresponding to the region R1 is highly likely to be present.

Here, the setting function 353 can set the segmentation region using classification results and anatomical landmarks obtained in the segmentation process described below. For example, the setting function 353 sets a segmentation region based also on a result classified as the liver and on a proximity to the anatomical landmarks of the liver.

### Segmentation Process

As described with step S105 in FIG. 2, the processing function 354 performs segmentation on the segmentation region set by the setting function 353. Specifically, the processing function 354 performs segmentation by extracting the segmentation region from the medical image and inputting the extracted segmentation region to the trained model constructed by deep learning.

Here, the trained model described above is constructed to output multi-class classification results and segmentation results. For example, the trained model described above may be constructed by Mask Region-Convolutional Neural Network (R-CNN) that performs both classification of class names of structures and per-pixel classification in the image in parallel. The above-described example is only an example, and other methods may be used to construct the trained model. A trained model for classifying class names of structures and a trained model for performing per-pixel region extraction may also be constructed separately and used.

### Process for Determining Structure

As described with step S106 in FIG. 2, the acquisition function 352 determines whether or not the structure serving as a target for segmentation is present outside the segmentation region. Specifically, the acquisition function 352 determines, on the basis of information on the existence probability, whether or not the structure serving as a target is connected to outside of the segmentation region. More specifically, the acquisition function 352 determines the connection between sub-regions among a plurality of the sub-regions on the basis of at least one of the structure continuity, distribution of the probability map of the structure, or anatomical landmarks.

For example, when the structure continuity has been acquired as information on the existence probability, the acquisition function 352 determines, in a case where similarity between pixel values of pixels outside the region closest to the segmented region and pixel values of pixels included in the extracted region by the segmentation is equal to or more than a threshold, that the structure serving as a target is present outside the segmentation region. In contrast, the acquisition function 352 determines, in a case where similarity between the pixel values of the pixels outside the region and the pixel values of the pixels included in the extracted region by the segmentation is less than the threshold, that the structure serving as a target is not present outside the segmentation region.

For example, when the probability map has been acquired as information on the existence probability, the acquisition function 352 determines, in a case where the probability map value of the outermost pixel within the segmentation region is equal to or more than a threshold, that the structure serving as a target is present outside the segmentation region. In contrast, in a case where the probability map value of the outermost pixel within the segmentation region is less than the threshold value, the acquisition function 352 determines that the structure serving as a target is not present outside the segmentation region.

For example, when the anatomical landmarks are acquired as information on the existence probability, the acquisition function 352 determines, on the basis of the anatomical landmarks, whether or not the structure serving as a target is present outside the segmentation region from an arrangement state of the estimated structure.

The acquisition function 352 performs the above-described determination process for the entire periphery of the segmentation region. For example, in a case where the segmentation region is a rectangle as illustrated in FIG. 4, the acquisition function 352 performs the above-described process for determining on each side of the segmentation region.

### Process for Displaying Segmentation Result

As described with step S107 in FIG. 2, the control function 351 causes the display 33 to display the result of the segmentation process executed by the processing function 354. Specifically, the control function 351 causes the display 33 to display the structure extracted by the process illustrated in FIG. 2. For example, the control function 351 causes the extracted structure to be highlighted in the display image generated from the medical image.

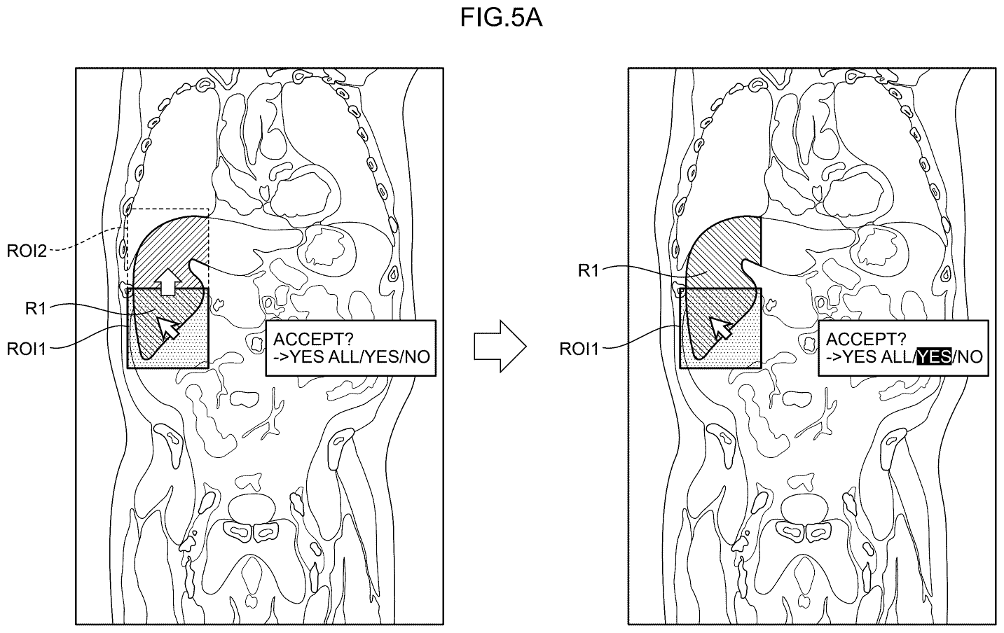

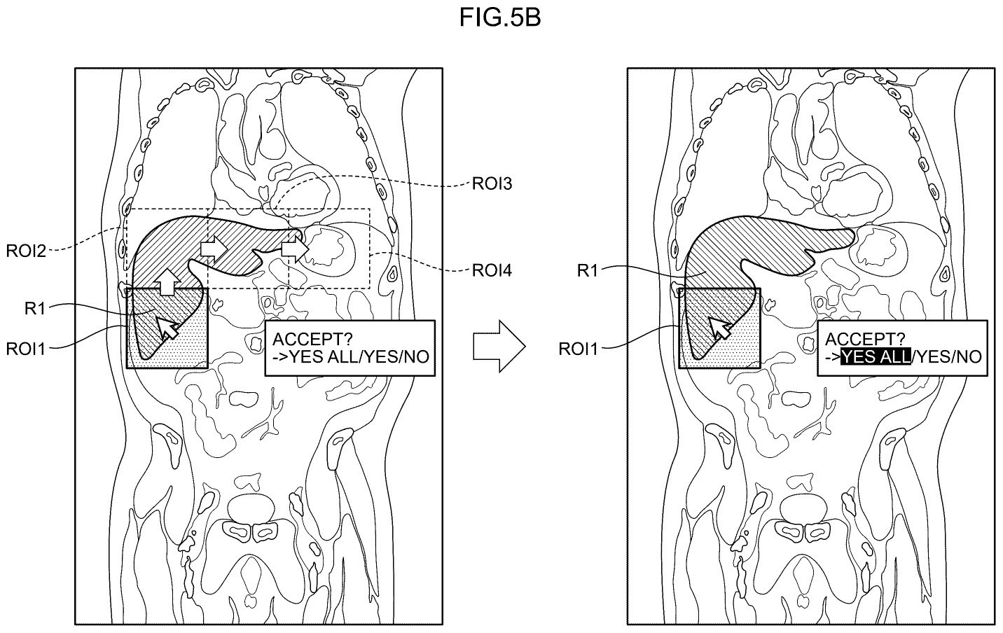

Hereinafter, an example of processing performed by the medical image processing apparatus 3 will be described with reference to FIGS. 5A and 5B. FIGS. 5A and 5B are diagrams, each illustrating an example of the processing executed by the medical image processing apparatus 3 according to the first embodiment. Here, FIGS. 5A and 5B illustrate the post-segmentation process illustrated in FIG. 4. In other words, FIGS. 5A and 5B illustrate the process after the segmentation region ROI1 (a sub-region constituting a part of the entire segmentation region) is set by the operator and the region R1 is extracted from the segmentation region ROI1 will be described.

As illustrated in FIG. 5A, once the region R1 is extracted by the processing function 354, the acquisition function 352 determines whether or not the structure corresponding to the region R1 is present outside the segmentation region ROI1. In other words, the acquisition function 352 performs the process at step S106 in FIG. 2. Here, the acquisition function 352 determines that the structure is present in an upper region of the segmentation region ROI1 in the figure, and acquires information on the existence probability of the structure for the upper region of the segmentation region ROI1.

The setting function 353 sets a segmentation region ROI2 illustrated in FIG. 5A on the basis of the information on the existence probability acquired by the acquisition function 352. Here, the medical image processing apparatus 3 can automatically perform segmentation on the segmentation region ROI2 set by the setting function 353. However, as illustrated in FIG. 5A, the medical image processing apparatus 3 can also receive an instruction from the operator as to whether or not to approve the segmentation region ROI2 as the segmentation region.

For example, the control function 351 displays the segmentation region in the medical image and a GUI for accepting instructions from the operator, as illustrated in the left diagram in FIG. 5A. Then, as illustrated in the right diagram in FIG. 5A, in a case where the segmentation region ROI2 is approved, the processing function 354 performs the segmentation process on the segmentation region ROI2 and extracts a structure serving as a target within the segmentation region ROI2. Therefore, as illustrated in the right diagram in FIG. 5A, the region R1 corresponding to the structure serving as a target is further extracted.

When the region R1 is further extracted as illustrated in the right diagram in FIG. 5A, the acquisition function 352 again determines whether or not the structure serving as a target is present outside the segmentation region. In other words, the acquisition function 352 determines whether or not the structure corresponding to the region R1 outside the segmentation region ROI2. In this way, the medical image processing apparatus 3 enables suppression of enlargement of a target region for segmentation and efficient performance of highly accurate segmentation by repeatedly performing determination of presence or absence of the structure serving as a target and the segmentation process using sub-regions.

In FIG. 5A, although a case has been described in which an instruction is received from the operator as to whether or not to approve setting of the sub-region for each setting of the sub-region, the embodiment is not limited thereto, and it is also possible to receive approval from the operator after a plurality of the sub-regions for extracting the entire structure serving as a target are set.

For example, after a region R1 is extracted by the processing function 354, the medical image processing apparatus 3 sequentially performs setting of the segmentation region ROI2 and the segmentation process within the ROI2, setting of a segmentation region ROI3 and the segmentation process within the ROI3, and setting of a segmentation region ROI4 and the segmentation process within the ROI4, as illustrated in FIG. 5B. Thereafter, the medical image processing apparatus 3 can be controlled to accept or reject approval for these processes from the operator.

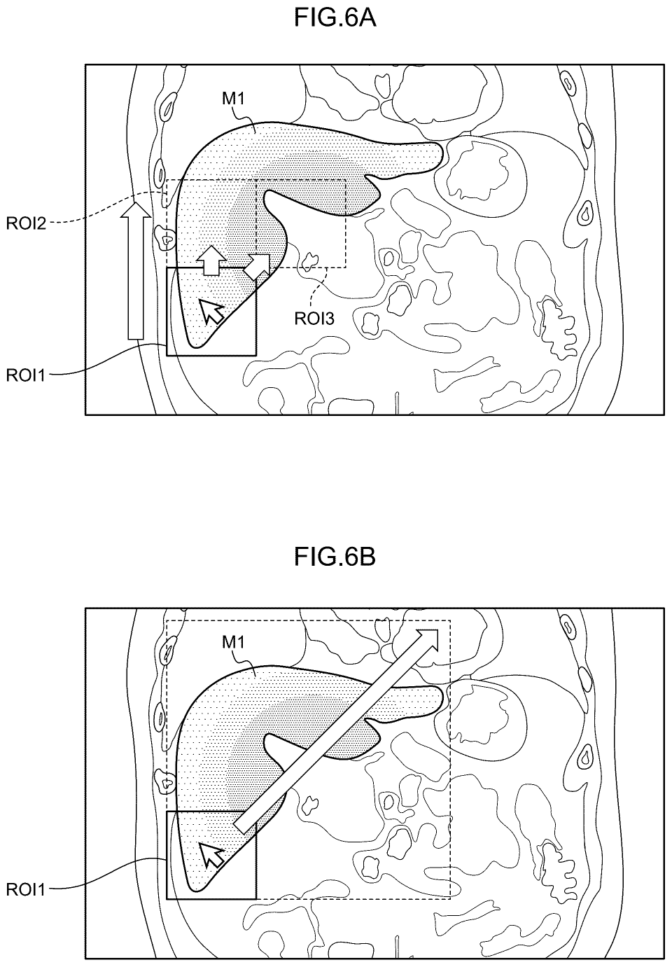

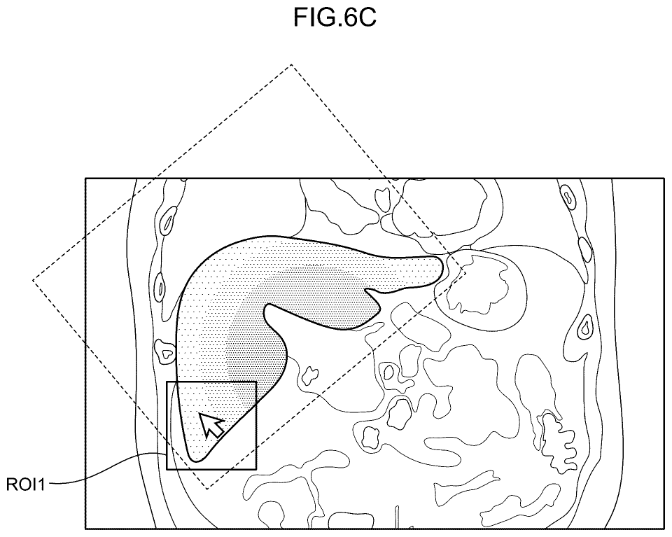

The processes described in FIGS. 5A and 5B are only an example, and the medical image processing apparatus 3 can perform various other processes. For example, the probability map is acquired as information on the existence probability, and the probability map value is used to set the segmentation regions, as illustrated in FIGS. 6A to 6C. FIGS. 6A to 6C diagrams, each illustrating an example of setting the segmentation regions using the probability map according to the first embodiment.

For example, as illustrated in FIG. 6A, in a case where segmentation is performed on the segmentation region ROI1, the setting function 353 can set the segmentation region ROI2, the segmentation region ROI3, and the like on the basis of the gradient of a probability map M1 in the segmentation region ROI1. In other words, the setting function 353 can set a new segmentation region (new sub-region) in a direction in which the probability increases in the gradient of the probability map M1 within the segmentation region ROI1

For example, in a case where segmentation is performed on the segmentation region ROI1 and the average of values of the probability map M1 within the segmentation region ROI1 is low, the setting function 353 can enlarge and set the segmentation region ROI1, as illustrated in FIG. 6B. That is, in a case where the average of values of a probability map M1 is low, the setting function 353 determines that the initial segmentation region ROI1 is close to an end of the structure, and can enlarge and reset the segmentation region ROI1.

Here, in a case where segmentation is performed on the segmentation region ROI1 and the probability map M1 within the segmentation region ROI1 is obtained, the setting function 353 can set the enlarged segmentation region ROI1 on the basis of the gradient of the probability map M1, and can also change an orientation of the segmentation region ROI1 in this case. For example, the setting function 353 can set the rotated and enlarged segmentation region ROI1 so that one segmentation region covers the structure serving as a target, as illustrated in FIG. 6C.

Here, the setting function 353 can also use information such as information on anatomical landmarks. In other words, the setting function 353 can set the orientation and size of the segmentation region ROI1 using information on anatomical landmarks in addition to the probability map. The above-described example is only an example, and the setting function 353 can flexibly set a segmentation region using information on the existence probability. For example, the setting function 353 can set the shape, size, and orientation of the segmentation region on the basis of at least one of structure continuity, distribution of the probability map of the structure, or anatomical landmarks.

The above-described embodiment describes a case in which the segmentation region is set using the probability map of the structure serving as a target for segmentation. However, the embodiment is not limited thereto, and in setting of a segmentation region, a probability map of a structure other than the structure serving as a target for segmentation may also be used.

For example, when setting the segmentation regions ROI2 to ROI4 illustrated in the left diagram in FIG. 5B, the setting function 353 also refers to a probability map of structures other than the structure (organ) corresponding to the region R1 to set a segmentation region. Here, in the segmentation region ROI4 in the left diagram of FIG. 5B, the probability of structures other than the structure (organ) corresponding to the region R1 increases. Therefore, the setting function 353 sets only the segmentation regions ROI2 and ROI3 and does not set the segmentation region ROI4.

The above-described embodiment also describes the point that a plurality of pieces of information on the existence probability can be used to set the segmentation regions. For example, the segmentation regions can be set using the anatomical landmarks and the probability map; however, inconsistencies may occur between the information on the anatomical landmarks and the information on the probability map. In one example, the liver may not be inferred as a classification result in a region containing anatomical landmarks of the liver. In such a case, the processing function 354 lowers the threshold of the probability map and performs segmentation again. The setting function 353 sets the next segmentation region using the probability map obtained from the segmentation performed again.

In the above-described embodiment, a case where the presence or absence of the structure outside the segmentation region is determined on the basis of information on the existence probability, and the next segmentation region is set on the basis of the determination result has been described. However, the embodiment is not limited thereto, and a case where the segmentation region is set on the basis of a shape of a region of the structure at a boundary of the segmentation region may also be employed.

For example, in the left diagram of FIG. 5A, at the upper boundary of the segmentation region ROI1, the shape of the region R1 substantially coincides with the shape of segmentation region ROI1. In other words, the organ corresponding to the region R1 is in a state of being cut in the segmentation region ROI1, and in that direction, it is highly likely that the organ is not fully included within the segmentation region ROI1. Accordingly, the setting function 353 may calculate a degree of match between the shape of the structure at the boundary of the segmentation region and the shape of the segmentation region, and set the segmentation region in a case where the degree of match is more than a threshold.

In the above-described embodiment, the case where the cursor associated with the segmentation region is used in the setting of the initial segmentation region has been described. However, the embodiment is not limited thereto, and the initial segmentation region may be set by other methods.

For example, the application of object detection to the setting of the initial segmentation region may be adopted. In such a case, the operator operates a cursor not associated with the segmentation region to designate the structure serving as a target for segmentation. The setting function 353 replaces a position designated by the cursor (for example, a clicked point) with an anchor for foreground/background classification in the object detection, and outputs a bounding box (BBox) from the clicked point to set it as a segmentation region.

For example, in the setting of the initial segmentation region, the position may not be designated by the operator. In other words, the information on the structure serving as a target for segmentation may be acquired from other information instead of the designation operation by the operator. In such a case, the control function 351 acquires the structure serving as a target for segmentation on the basis of input information on the structure.

For example, the control function 351 acquires information on the structure serving as a target for segmentation on the basis of scan conditions for collecting medical images and information input by the operator (for example, the name of an organ). The setting function 353 sets a segmentation region on the medical image on the basis of information of the target for segmentation acquired by the control function 351.

Here, the memory 34 stores, in advance, correspondence information in which a setting position of a segmentation region is associated with each organ. For example, the correspondence information is information in which a setting position is defined for each organ using positional information represented by positions of anatomical landmarks. The setting function 353 acquires a setting position of an initial segmentation by acquiring the correspondence information on the target for segmentation acquired by the control function 351. Furthermore, the setting function 353 detects anatomical landmarks in the medical image by performing image processing on the medical image, and sets the segmentation region on the basis of the detected anatomical landmarks and the setting position of the initial segmentation.

In the embodiment described above, the case where segmentation is performed for a single structure (liver) has been described, but the embodiment is not limited thereto, and segmentation for extracting a plurality of structures may also be performed. For example, in a case where the structures such as ribs or vertebrae are included, one or more structures may be extracted depending on operations of the operator.

As one example, segmentation may be performed to extract all vertebrae when the operator sets an initial segmentation region to include a plurality of vertebral bodies. On the other hand, in a case where the operator sets the initial segmentation region to include only a single vertebral body, segmentation may be performed to extract only the designated vertebra.

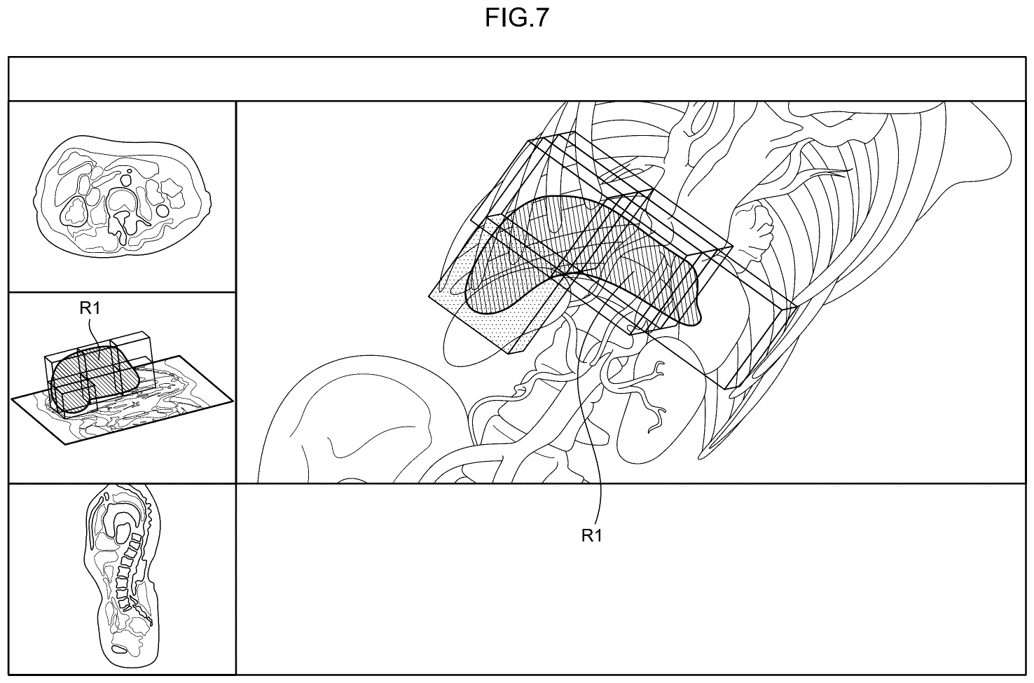

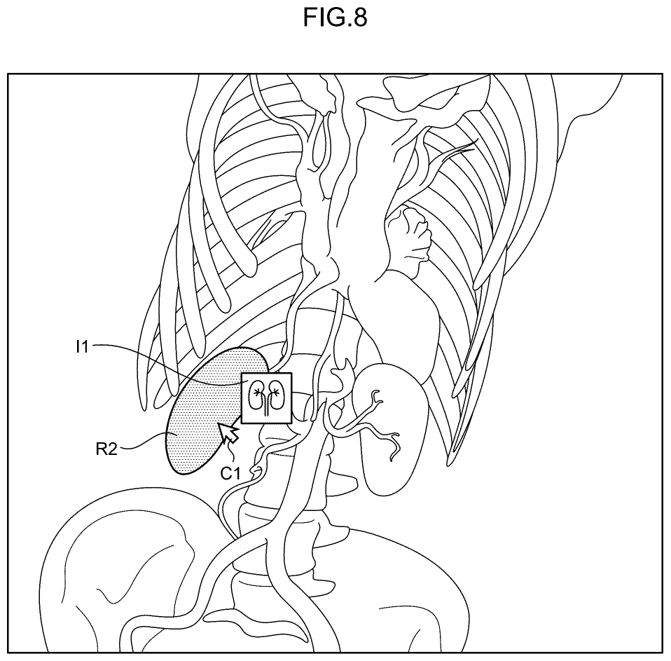

The case in which the segmentation region is set on a two-dimensional display image has been described, but the embodiment is not limited thereto, and the segmentation region can also be displayed in three dimensions. FIG. 7 is a diagram illustrating an example of display according to the first embodiment. Here, in FIG. 7, MPR images of three orthogonal cross sections are illustrated on the left side, and a three-dimensional image is illustrated on the right side.

For example, as illustrated in FIG. 7, the control function 351 can cause the segmentation region to be displayed in three dimensions by tilting the image in which the segmentation region is set within the MPR images of three orthogonal cross sections. In addition, as illustrated in FIG. 7, the control function 351 can cause the segmentation region to be displayed in three dimensions by placing the segmentation region on the three-dimensional image. By displaying the segmentation region in three dimensions, the extracted region R1 can also be observed in three dimensions.

In the embodiment described above, highlighting the extracted structure in the display of the segmentation result has been exemplified. However, the embodiment is not limited thereto, and other information may be displayed. FIG. 8 is a diagram illustrating an example of display of the segmentation result according to the first embodiment.

For example, as illustrated in FIG. 8, the control function 351 can highlight the organ designated by the cursor C1 and also display the classification result of segmentation as an icon or the like. In other words, in the medical image processing apparatus 3, when the operator performs an operation to designate the target for segmentation (for example, a click) while moving the cursor C1, the target organ is highlighted, and information on classification results can be displayed.

As described above, according to the first embodiment, the control function 351 acquires information on the structure serving as a target for segmentation. The acquisition function 352 acquires information on the existence probability of a structure in a medical image on the basis of information on the structure. The setting function 353 sets a segmentation region for performing segmentation of the structure on a part of the medical image on the basis of the information on the existence probability of the structure. The processing function 354 performs segmentation of the structure with respect to the segmentation region of the medical image. Therefore, the medical image processing apparatus 3 according to the first embodiment allows the operator to perform segmentation only on the desired structure, and it is possible to perform highly accurate segmentation efficiently.

For example, since the processing range can be restricted to only the target desired by the operator, it is possible to perform local bone segmentation or local vessel segmentation, and unnecessary portions can be removed in three-dimensional display, or the associated computational cost can be reduced.

Furthermore, for example, since an independent region such as a fractured region can be easily extracted, the medical image processing apparatus 3 can automatically display the fractured region in a cross-section that facilitates observation thereof. The medical image processing apparatus 3 can also extract a single vertebral body and perform labeling on the extracted vertebral body based on information on surrounding anatomical landmarks.

For example, since the medical image processing apparatus 3 performs segmentation based on deep learning, it is possible to perform segmentation with high accuracy even within regions where similar pixel values are contiguous.

Furthermore, according to the first embodiment, the acquisition function 352 acquires, as information on the existence probability of the structure, at least one of structure continuity, a probability map, and an anatomical landmark. Therefore, the medical image processing apparatus 3 according to the first embodiment can accurately determine the presence or absence of the structure.

Furthermore, according to the first embodiment, the acquisition function 352 acquires, as the probability map, at least one of a probability map of the structure or a probability map of structures other than the structure. Therefore, the medical image processing apparatus 3 according to the first embodiment can appropriately determine the presence or absence of the structure.

Furthermore, according to the first embodiment, the control function 351 acquires the structure serving as a target for segmentation on the basis of a designation operation by an operator on the medical image. Therefore, the medical image processing apparatus 3 according to the first embodiment can easily acquire information on the structure serving as a target for segmentation.

According to the first embodiment, the control function 351 acquires the structure serving as a target for segmentation on the basis of the input information of the structure. Therefore, the medical image processing apparatus 3 according to the first embodiment can acquire information on the structure serving as a target for segmentation without the operator performing a designation operation.

According to the first embodiment, the setting function 353 can set the shape, size, and orientation of the segmentation region on the basis of at least one of structure continuity, distribution of the probability map of the structure, or anatomical landmarks. Therefore, the medical image processing apparatus 3 of the first embodiment can set segmentation regions according to various situations.

According to the first embodiment, the segmentation region includes a plurality of sub-regions, and the acquisition function 352 determines the connection between sub-regions among the sub-regions on the basis of at least one of the structure continuity, distribution of the probability map of the structure, or anatomical landmarks. Therefore, the medical image processing apparatus 3 according to the first embodiment can finely determine connectivity of the structure and can suppress enlargement of the segmentation region.

In addition, according to the first embodiment, the control function 351 causes the segmentation region to be displayed on the medical image. Therefore, the medical image processing apparatus 3 according to the first embodiment can cause the operator to confirm the set segmentation region.

### Other Embodiment

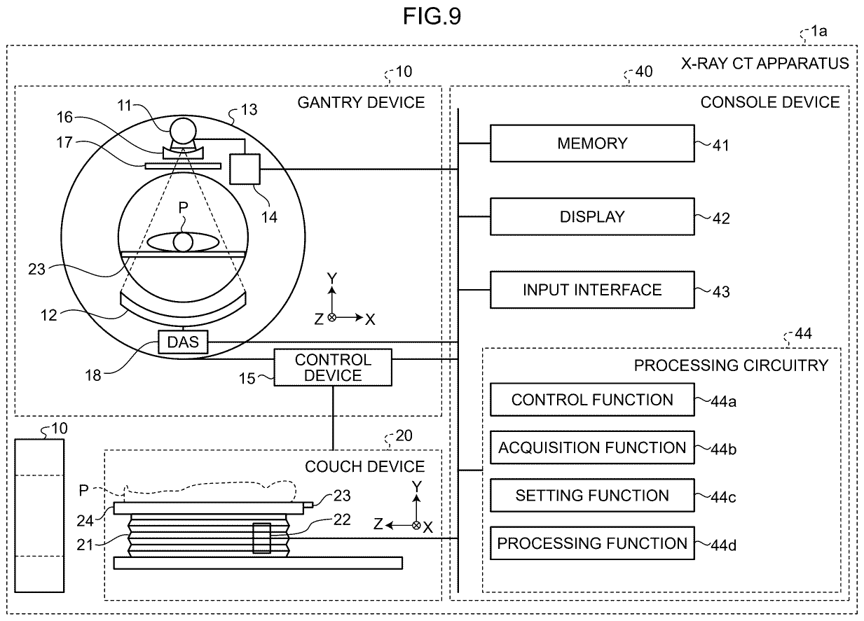

In the first embodiment described above, the case in which the medical image processing apparatus 3 performs each of the processes according to the present application has been described. However, the medical image diagnostic apparatus 1 may perform each of the processes according to the present application. FIG. 9 is a diagram illustrating a configuration example of an X-ray CT apparatus 1a according to another embodiment. For example, the X-ray CT apparatus 1a includes a gantry device 10, a couch device 20, and a console device 40.

In FIG. 9, the rotational axis of a rotating frame 13 in a non-tilt state or the longitudinal direction of a couchtop 23 of the couch device 20 is defined as the Z-axis direction. The axis direction that is orthogonal to the Z-axis direction and horizontal with respect to the floor surface is defined as the X-axis direction. The axial direction that is orthogonal to the Z-axis direction and perpendicular to the floor surface defined as the Y-axis direction. FIG. 9 illustrates a case where the X-ray CT apparatus 1a has one gantry device 10, with the gantry device 10 illustrated from multiple directions for illustrative purposes.

The gantry device 10 includes an X-ray tube 11, an X-ray detector 12, a rotating frame 13, an X-ray high-voltage device 14, a control device 15, a wedge 16, a collimator 17, and a data acquisition system (DAS) 18.

The X-ray tube 11 is a vacuum tube including a cathode (filament) that generates thermoelectrons and an anode (target) that generates X-rays upon receiving collisions of the thermoelectrons. The X-ray tube 11 generates X-rays with which a subject P is irradiated by emitting thermoelectrons from the cathode toward the anode upon application of a high voltage from the X-ray high-voltage device 14.

The X-ray detector 12 detects X-rays emitted from the X-ray tube 11 and passing through the subject P, and outputs a signal corresponding to the detected X-ray dose to the DAS 18. The X-ray detector 12 includes, for example, a plurality of detector element arrays in which a plurality of detector elements are arranged in a channel direction along a single arc centered on a focal point of the X-ray tube 11. The X-ray detector 12 has, for example, a structure in which the detector element arrays, each having the detection elements arranged in the channel direction, are arranged in a row direction (slice direction). The X-ray detector 12 is, for example, an indirect-conversion type detector having a grid, a scintillator array, and a photodetector array. The scintillator array includes multiple scintillators. A scintillator has a scintillator crystal that outputs a photon amount of light corresponding to the incident X-ray dose. The grid has an X-ray shielding plate that is placed on an X-ray incidence surface of the scintillator array and absorbs scattered X-rays. The grid may also be referred to as a collimator (one-dimensional collimator or two-dimensional collimator). The photodetector array has a function of converting the amount of light from the scintillator into an electric signal, and includes, for example, a photodetector such as a photodiode. The X-ray detector 12 may be a direct conversion type detector with semiconductor elements that convert the incident X-rays into electrical signals.

The rotating frame 13 is an annular frame that supports the X-ray tube 11 and the X-ray detector 12 to face each other, and rotates the X-ray tube 11 and the X-ray detector 12 under the control of the control device 15. For example, the rotating frame 13 is cast aluminum material. In addition to the X-ray tube 11 and the X-ray detector 12, the rotating frame 13 may further support the X-ray high-voltage device 14, the wedge 16, the collimator 17, the DAS 18, and other components.

The control device 15 controls the operations of the gantry device 10 and the couch device 30. The wedge 16 is an X-ray filter for adjusting the X-ray dose emitted from the X-ray tube 11. The collimator 17 is an X-ray diaphragm that narrows the irradiation range of X-rays having passed through the wedge 16. The narrowing range of the collimator 17 may be operated mechanically.

The DAS 18 collects X-ray signals detected by the individual detection elements included in the X-ray detector 12. For example, the DAS 18 includes an amplifier that amplifies electric signals output from the individual detection elements and an A/D converter that converts the electric signals into digital signals, and generates detection data.

The data generated by the DAS 18 is transmitted via optical communication from a transmitter including a light-emitting diode (LED) and provided on the rotating frame 13 to a receiver including a photodiode and provided on a non-rotating part of the gantry device 10, and is then transferred to the console device 40. Here, the non-rotating part refers to, for example, a fixed frame that rotatably supports the rotating frame 13, and the like. A data transmission method from the rotating frame 13 to the non-rotating part of the gantry device 10 is not limited to optical communication and may employ any non-contact type data transmission method or a contact type data transmission method. The X-ray detector 12 and the DAS 18 may be formed as an integrated detector unit (DU).

The couch device 20 is a device for placing and moving the subject P to be subjected to CT scanning, and includes a base 21, a couch driving device 22, a couchtop 23, and a support frame 24. The base 21 is a housing that supports the support frame 24 to be movable in the vertical direction. The couch driving device 22 is a drive mechanism that moves the couchtop 23, on which the subject P is placed, in the longitudinal direction of the couchtop 23, and includes a motor, an actuator, and the like. The couchtop 23 on the top surface of the support frame 24 is a plate on which the subject P is placed. The couch driving device 22 may move not only the couchtop 23 but also the support frame 24 in the longitudinal direction of the couchtop 23.

The console device 40 includes a memory 41, a display 42, an input interface 43, and processing circuitry 44. Although the console device 40 is described as being separate from the gantry device 10, the console device 40 or part of the constituent elements of the console device 40 may be included in the gantry device 10.

The memory 41 may be implemented by, for example, semiconductor memory devices such as random access memory (RAM) or flash memory, a hard disk, an optical disk, or the like. For example, the memory 41 stores projection data collected by CT scanning and X-ray CT images reconstructed on the basis of the projection data. The memory 41 stores a computer program for enabling circuits included in the X-ray CT apparatus 1a to implement functions thereof. The memory 41 may be implemented by a group of servers (cloud) connected to the X-ray CT apparatus 1a via a network.

The display 42 displays various types of information under the control of the processing circuitry 44. For example, the display 42 displays a graphical user interface (GUI) for receiving various instructions, settings, and the like from a user via the input interface 43. The display 42 also displays a display image generated on the basis of the X-ray CT image. For example, the display 42 may be a liquid crystal display or a cathode ray tube (CRT) display. The display 42 may be a desktop type, or may alternatively be configured as a tablet terminal or the like capable of wireless communication with the processing circuitry 44.

The input interface 43 receives various input operations from a user, converts the received input operations into electric signals, and outputs the signals to the processing circuitry 44. For example, the input interface 43 may be implemented by a mouse, a keyboard, a trackball, a switch, a button, a touch pad that receives input operations through for input operations in response to a touch on an operation surface, a touch screen in which a display screen and a touch pad are integrated, a non-contact input circuit using an optical sensor, a voice input circuit, or the like. The input interface 43 may be configured as a tablet terminal or the like capable of wireless communication with the processing circuitry 44. The input interface 43 may be a circuit that receives input operations from the user through motion capture. As one example, the input interface 43 can receive, as input operations, body movements or eye gaze of the user by processing signals acquired via a tracker or images collected of the user. In addition, the input interface 43 is not limited only to those with physical operating components such as a mouse and a keyboard. For example, an electrical signal processing circuit that receives electrical signals corresponding to input operations input from an external input device provided separately from the X-ray CT apparatus 1a and outputs these electrical signals to the processing circuitry 44 is also an example of the input interface 43.

The processing circuitry 44 controls the overall operation of the X-ray CT apparatus 1a by executing a control function 44a, an acquisition function 44b, a setting function 44c, and a processing function 44d. For example, the processing circuitry 44 functions as the control function 44a by reading and executing a computer program corresponding to the control function 44a from the memory 41. Accordingly, the processing circuitry 44 functions as the acquisition function 44b, the setting function 44c, and the processing function 44d. The processing circuitry 44 is an example of a processing circuitry.

For example, the control function 44a controls the operations of the gantry device 10 and the couch device 20 in accordance with instructions from the user received via the input interface 43, and performs CT scanning on the subject P.

For example, the control function 44a supplies a high voltage to the X-ray tube 11 by controlling the X-ray high-voltage device 14. As a result, the X-ray tube 11 generates X-rays with which the subject P is irradiated. The control function 44a moves the subject P into an imaging opening of the gantry device 10 by controlling the couch driving device 22. The control function 44a controls the distribution of X-rays with which the subject P is irradiated by adjusting a position of the wedge 16 and an aperture and a position of the collimator 17.

The control function 44a controls the X-ray detector 12 and the DAS 18 to detect X-rays emitted from the X-ray tube 11 and collect detection data. The control function 44a may also perform various types of processing based on detection data collected by CT scanning. For example, the control function 44a performs preprocessing on the detection data output from the DAS 18, such as logarithmic conversion processing, offset correction processing, inter-channel sensitivity correction processing, beam hardening correction, scatter correction, and dark count correction. The detection data after the preprocessing is also referred to as raw data. The detection data before preprocessing and the raw data after preprocessing are collectively referred to as projection data. Furthermore, the control function 44a generates an X-ray CT image by performing reconstruction processing on the projection data using a filtered back-projection method, an iterative reconstruction method, or the like. Various types of data, such as projection data and X-ray CT images, are appropriately stored in the memory 41. The projection data and the X-ray CT images are collectively referred to as CT images.

The control function 44a performs processing similar to the control function 351 described above. The acquisition function 44b performs processing similar to the acquisition function 352 described above. The setting function 44c performs processing similar to the setting function 353 described above. The processing function 44d performs processing similar to the processing function 354 described above.

In the X-ray CT apparatus 1a illustrated in FIG. 9, each processing function is stored in the memory 41 in the form of a computer-executable program. The processing circuitry 44 is a processor that reads computer programs from the memory 41 and executes each computer program to implement a function corresponding to each computer program. In other words, the processing circuitry 44, in a state where each computer program has been read out, has the function corresponding to the read program.

In FIG. 9, although the X-ray CT apparatus 1a has been described as an example of the medical image diagnostic apparatus 1 that executes each processing according to the present application, the embodiment is not limited thereto. That is, the medical image diagnostic apparatus 1 other than the X-ray CT apparatus 1a may also execute each processing according to the present application. For example, an X-ray diagnostic apparatus, an MRI apparatus, an ultrasound diagnostic apparatus, a SPECT apparatus, a PET apparatus, or the like may execute each processing according to the present application.

The processing circuitry described in each embodiment described above may be configured by combining a plurality of independent processors, each of which implements each processing function by executing a computer program. Each processing function of the processing circuitry may be appropriately distributed across or integrated into one or more processing circuits. Each processing function of the processing circuitry may also be implemented by a combination of hardware, such as circuits, and software. Although an example in which the computer programs corresponding to the respective processing functions are stored in the single storage circuit or memory has been described, the embodiment is not limited thereto. For example, the computer programs corresponding to the respective processing functions may be distributed and stored across a plurality of the memories, and the processing circuitry may be configured to read and execute each program from the respective storage circuits or memories.

In the embodiment described above, an example in which each part in the present specification is implemented by each function of the processing circuitry has been described; however, the embodiment is not limited thereto. For example, each part in the present specification may be implemented not only by the respective functions described in the embodiment, but also by hardware alone, software alone, or a combination of hardware and software.

The term “processor” described in the above-described embodiment means, for example, a circuitry such as a central processing unit (CPU), a graphics processing unit (GPU), an application specific integrated circuit (ASIC), or a programmable logic device (for example, simple programmable logic device (SPLD), complex programmable logic device (CPLD), and field programmable gate array (FPGA)). Here, instead of storing the computer programs in the memory, the computer programs may be configured to be incorporated directly into the circuit of the processor. In this case, the processor reads and executes the computer programs incorporated in the circuit to execute the functions thereof. Each processor of the present embodiment is not limited to the configuration of a single circuit provided for each processor, and may also employ the configuration of a single processor including a plurality of independent circuits in combination to execute the functions thereof.

Here, a medical image processing program executed by a processor is provided with the computer program being pre-embedded in a read only memory (ROM), a memory circuit, or other memories. This medical image processing program may be stored and provided in a format that can be installed in these apparatuses or as a file in an executable format in a non-transitory computer readable medium, such as a compact disc (CD)-ROM, flexible Disk (FD), CD-recordable (R), digital versatile disc (DVD), or other storage medium. This medical image processing program may also be stored in a computer connected to a network, such as the Internet, and may be provided or distributed by downloading via the network. For example, this medical image processing program includes modules that have processing functions described above. As for the actual hardware, the CPU reads and executes the medical image processing program from a storage medium such as a ROM, and each module is loaded in a main memory device and generated in the main memory device.

Each of the components of each of the apparatuses illustrated in the above-described embodiments and modifications is a functional concept, and does not necessarily have the physical configuration as illustrated in the figures. That is, the specific form of distribution and integration of each of the apparatuses is not limited to those illustrated in the figures, and all or parts thereof can be functionally or physically distributed and integrated in any units according to various loads and usage conditions. Furthermore, each of the processing functions performed by each of the apparatuses can be implemented, in all or any parts thereof, by CPU and a computer program analyzed and executed by the CPU, or by hardware using wired logic.

Among the various types of processing described in the above-described embodiments and modifications, all or part of the processing described as being performed automatically may instead be performed manually, and conversely, all or part of the processing described as being performed manually may be performed automatically using known methods. In addition, unless otherwise specified, the processing procedures, control procedures, specific names, and information including various data and parameters described in the above-described specification and drawings can be optionally modified.

According to at least one of the embodiments described above, it is possible to efficiently perform highly accurate segmentation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising processing circuitry configured to
acquire information on a structure serving as a target for segmentation,
acquire information on an existence probability of the structure in a CT image on a basis of the information on the structure,
set a segmentation region where segmentation of the structure is performed on a part of the CT image on a basis of the information on the existence probability of the structure, and
perform segmentation of the structure on the segmentation region of the CT image.

2. A medical image processing apparatus comprising processing circuitry configured to
acquire information on a structure serving as a target for segmentation,
acquire information on an existence probability of the structure in a medical image on a basis of the information on the structure,
set a segmentation region where segmentation of the structure is performed on a part of the medical image on a basis of the information on the existence probability of the structure, and
perform segmentation of the structure on the segmentation region of the medical image.

3. The medical image processing apparatus according to claim 2, wherein
the processing circuitry is configured to acquire at least one of structure continuity, a probability map, and an anatomical landmark as information on the existence probability of the structure.

4. The medical image processing apparatus according to claim 3, wherein
the processing circuitry is configured to acquire, as the probability map, at least one of a probability map of the structure and a probability map of a structure other than the structure.

5. The medical image processing apparatus according to claim 2, wherein
the processing circuitry is configured to acquire the structure serving as a target for segmentation on a basis of a designation operation by an operator on the medical image.

6. The medical image processing apparatus according to claim 2, wherein
the processing circuitry is configured to acquire the structure serving as a target for segmentation on a basis of input information on a structure.

7. The medical image processing apparatus according to claim 2, wherein
the processing circuitry is configured to set a shape, a size, and an orientation of the segmentation region on a basis of at least one of structure continuity, distribution of a probability map of the structure, or an anatomical landmark.

8. The medical image processing apparatus according to claim 7, wherein
the segmentation region includes a plurality of sub-regions, and
the processing circuitry is configured to determine connection between sub-regions among the sub-regions on a basis of at least one of structure continuity, distribution of a probability map of the structure, or an anatomical landmark.

9. The medical image processing apparatus of claim 2, wherein
the processing circuitry is further configured to cause the segmentation region to be displayed in the medical image.

10. A method comprising:
acquiring information on a structure serving as a target for segmentation;
acquiring information on an existence probability of the structure in a medical image on a basis of the information on the structure;
setting a segmentation region where segmentation of the structure is performed on a part of the medical image on a basis of the information on the existence probability of the structure; and
performing segmentation of the structure on the segmentation region of the medical image.

11. A non-transitory computer readable medium comprising instructions that cause a computer to execute:
acquiring information on a structure serving as a target for segmentation;
acquiring information on an existence probability of the structure in a medical image on a basis of the information on the structure;
setting a segmentation region where segmentation of the structure is performed on a portion of the medical image on a basis of the information on the existence probability of the structure; and
performing segmentation of the structure on the segmentation region of the medical image.
